(19) United States
(12) Patent Application Publication
(10) Pub. No.: US 2024/0279645 A1
(43) Pub. Date: Aug. 22, 2024

(54) METHODS AND COMPOSITIONS RELATED TO A MODIFIED METHYLTRANSFERASE

(71) Applicant: BOARD OF REGENTS, THE UNIVERSITY OF TEXAS SYSTEM, Austin, TX (US)

(72) Inventors: Andrew ELLINGTON, Austin, TX (US); Simon D'OELSNITZ, Austin, TX (US); Kamyab JAVANMARDI, Austin, TX (US); Ross THYER, Houston, TX (US)

(21) Appl. No.: 18/566,018

(22) PCT Filed: Jun. 2, 2022
(86) PCT No.: PCT/US2022/031986

§ 371 Date: Nov. 30, 2023

Related U.S. Application Data
(60) Provisional application No. 63/195,997, filed on Jun. 2, 2021.

(51) Int. Cl. C12N 15/10

(52) U.S. Cl. CPC C12N 15/1086; C12N 15/1072

(57) ABSTRACT

Disclosed herein are engineered methyltransferases which are capable of methylating more than two sites in a benzylisoquinoline alkaloid or precursor thereof. Also disclosed are of methods of making benzylisoquinoline alkaloids using the engineered methyltransferase. Lastly, disclosed are kits, nucleic acids, and proteins related to the methyltransferases disclosed herein.

### CROSS-REFERENCE TO RELATED APPLICATIONS

This application claims benefit of U.S. Provisional Application No. 63/195,997, filed Jun. 2, 2021, incorporated herein by reference in its entirety.

### GOVERNMENT SUPPORT CLAUSE

This invention was made with government support under Grant no. FA9550-14-1-0089 awarded by the Air Force Office of Scientific Research, and Grant no. HR0011-19-2-0019 awarded by the Defense Advanced Research Projects Agency (DARPA). The government has certain rights in the invention.

### BACKGROUND

Numerous benzylisoquinoline alkaloids (BIAs) have been recognized for their therapeutic value as modern pharmaceuticals. Beyond the well-known morphinans, the BIA scaffold is also used to produce tetrahydroisoquinoline neuromuscular-blocking agents and a vasodilator. Specifically, tetrahydropapaverine, which is naturally produced in plants, is used as a direct precursor to the four FDA-approved drugs atracurium, cisatracurium, mivacurium, and papaverine. In plants, THP is thought to be produced from the common intermediate norcoclaurine via an oxidase and four separate O-methyltransferases; however, some BIA methyltransferases have promiscuous activities and can methylate multiple positions.

What is needed in the art is a methyltransferase which is capable of methylating several positions in a BIA to reduce a multiple-enzyme pathway into one single enzyme.

### SUMMARY

Disclosed herein is a non-naturally occurring methyltransferase, wherein said methyltransferase can methylate more than two positions in a benzylisoquinoline alkaloid (BIA) molecule.

Also disclosed is a method of preparing a benzylisoquinoline alkaloid (BIA) composition, wherein the benzylisoquinoline alkaloid (BIA) composition requires methylation in its final form, the method comprising: culturing a host cell under suitable conditions, wherein the host cell comprises nucleic acid encoding a non-naturally occurring methyltransferase; exposing the methyltransferase to the precursor of the BIA composition; and allowing the methyltransferase to methylate the precursor to the BIA composition, wherein the methyltransferase methylates more than two positions in the precursor of the BIA composition, thereby producing a methylated composition of interest.

Further disclosed is a kit comprising: a non-naturally occurring methyltransferase, wherein said methyltransferase can methylate more than two positions in a benzylisoquinoline alkaloid (BIA) molecule.

### DESCRIPTION OF DRAWINGS

FIG. 1A-F shows performance of all top THP variants recovered. A) evolution of THP1. B) evolution of THP2. C) Evolution of THP3. D) Evolution of THP4. E) Selectivity of THP3 variants. F) Selectivity of THP4 variants.

FIG. 2A-B shows the characterization of the THP reporter plasmid (pThpR).

Dose response function of pThpR variants with different THP sensor variants and regulation types. “Auto” denotes that the THP sensor regulates its own expression whereas “P106” denotes that the THP sensor is constitutively expressed from the P106 anderson promoter. Both THP3 and THP4.1 were compared. The P106-THP4.1 variant of pThpR was used for subsequent fluorescence-based THP measurement assays. (b) The dose response of pThpR (P106-THP4.1) to norlaudanosoline, norreticuline, and tetrahydropapaverine.

FIG. 3 shows screening O-methyltransferases using pThpR. Cells expressing either an empty plasmid (TAA) or a BIA methyltransferase were co-transformed with pThpR and grown in the presence of 100 uM of norlaudanosoline and 1 mg/mL ascorbic acid for 18 hours at 30° C. and culture fluorescence was subsequently measured. Measurements were performed in biological triplicate.

FIG. 4A-D shows the evolution of an alkaloid methyltransferase via a sensor-linked screen. (a) Genetic scheme for O-methyltransferase (OMT) evolution. A plasmid expressing the OMT variant (blue) is co-transformed with a plasmid expressing GFP regulated by a tetrahydropapaverine-responsive biosensor (THP4.1). OMT libraries are plated with norlaudanosoline (NOR) and highly fluorescent colonies are picked, characterized, and the top variant is used for the following round of evolution. (b) Homology structure of the template OMT (GfOMT1) with mutations in evolved variants shown in orange and labelled. The substrate NOR and cofactor S-adenosyl-methionine are shown in pink and green respectively. (c) Fluorescence response of the THP4.1 reporter plasmid cultured with either 0, 10, or 100 uM of (NOR) and an empty plasmid (TAA), GfOMT1 (WT) or evolved OMT variants. (d) Representative ion extracted chromatograms of strains expressing engineered OMT variants, or controls, grown in the presence of 10 uM NOR. All LC-MS chromatograms were selected for the theoretical m/z values and retention times of the respective compounds of interest.

FIG. 5A-F shows performance of all top OMT variants recovered from each round of evolution. (a-e) Fluorescent response of top unique GLAU variants using the pThpR reporter. All variants were subcloned into a fresh pReg backbone prior to characterization with the pThpR plasmid. The “x4” symbol denotes that this amino acid sequence was recovered four times following evolution. The variant genotype highlighted in green was chosen as the template for the following round of evolution. Measurements were performed in biological triplicate or quadruplicate. (f) Concentration of the norlaudanosoline substrate used to characterize the performance of evolved OMTs in (a-e).

FIG. 6A-B shows local environment of W22L and I258V OMT mutations. A homology structure of the GEN5 OMT was constructed using SwissModel to infer the local environment of enzyme mutations. The substrate norlaudanosoline is shown in pink, the co-factor S-adenosyl-methionine is shown in green, and mutations are shown in orange. One monomer is transparent while the other monomer is colored blue. (a) Environment of the W22L mutation that appears in the GEN2 OMT variant. (b) Environment of the I258V mutations that appears in the GEN3 OMT variant.

FIG. 7 shows quantification of THP produced by each OMT variant. Cells expressing an empty plasmid (TAA) or an OMT variant were cultured in the presence of 10 uM NOR and 1 mg/mL ascorbic acid for 18 hours at 30 C and THP was quantified with LC/MS by fitting to a standard curve (R2=0.9999). Measurements were performed in biological triplicate.

### DETAILED DESCRIPTION

### General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur.

Reference is made herein to nucleic acid and nucleic acid sequences. The terms “nucleic acid” and “nucleic acid sequence” refer to a nucleotide, oligonucleotide, polynucleotide (which terms may be used interchangeably), or any fragment thereof. These phrases also refer to DNA or RNA of genomic or synthetic origin (which may be single-stranded or double-stranded and may represent the sense or the antisense strand).

Reference also is made herein to peptides, polypeptides, proteins and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

As disclosed herein, exemplary peptides, polypeptides, proteins may comprise, consist essentially of, or consist of any reference amino acid sequence disclosed herein, or variants of the peptides, polypeptides, and proteins may comprise, consist essentially of, or consist of an amino acid sequence having at least about 80%, 90%, 95%, 96%, 97%, 98%, or 99% sequence identity to any amino acid sequence disclosed herein. Variant peptides, polypeptides, and proteins may include peptides, polypeptides, and proteins having one or more amino acid substitutions, deletions, additions and/or amino acid insertions relative to a reference peptide, polypeptide, or protein. Also disclosed are nucleic acid molecules that encode the disclosed peptides, polypeptides, and proteins (e.g., polynucleotides that encode any of the peptides, polypeptides, and proteins disclosed herein and variants thereof).

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

Variants comprising deletions relative to a reference amino acid sequence or nucleotide sequence are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation or both of a reference polypeptide or a 5′-terminal or 3′-terminal truncation or both of a reference polynucleotide).

Variants comprising a fragment of a reference amino acid sequence or nucleotide sequence are contemplated herein. A “fragment” is a portion of an amino acid sequence or a nucleotide sequence which is identical in sequence to but shorter in length than the reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule, for example the N-terminal region and/or the C-terminal region of a polypeptide or the 5′-terminal region and/or the 3′ terminal region of a polynucleotide. The term “at least a fragment” encompasses the full-length polynucleotide or full-length polypeptide.

Variants comprising insertions or additions relative to a reference sequence are contemplated herein. The words “insertion” and “addition” refer to changes in an amino acid or nucleotide sequence resulting in the addition of one or more amino acid residues or nucleotides. An insertion or addition may refer to 1, 2, 3, 4, 5, 10, 20, 30, 40, 50, 60, 70, 80, 90, 100, 150, or 200 amino acid residues or nucleotides.

Fusion proteins and fusion polynucleotides also are contemplated herein. A “fusion protein” refers to a protein formed by the fusion of at least one peptide, polypeptide, protein or variant thereof as disclosed herein to at least one molecule of a heterologous peptide, polypeptide, protein or variant thereof. The heterologous protein(s) may be fused at the N-terminus, the C-terminus, or both termini. A fusion protein comprises at least a fragment or variant of the heterologous protein(s) that are fused with one another, preferably by genetic fusion (i.e., the fusion protein is generated by translation of a nucleic acid in which a polynucleotide encoding all or a portion of a first heterologous protein is joined in-frame with a polynucleotide encoding all or a portion of a second heterologous protein). The heterologous protein(s), once part of the fusion protein, may each be referred to herein as a “portion”, “region” or “moiety” of the fusion protein.

A fusion polynucleotide refers to the fusion of the nucleotide sequence of a first polynucleotide to the nucleotide sequence of a second heterologous polynucleotide (e.g., the 3′ end of a first polynucleotide to a 5′ end of the second polynucleotide). Where the first and second polynucleotides encode proteins, the fusion may be such that the encoded proteins are in-frame and results in a fusion protein. The first and second polynucleotide may be fused such that the first and second polynucleotide are operably linked (e.g., as a promoter and a gene expressed by the promoter as discussed below).

“Homology” refers to sequence similarity or, interchangeably, sequence identity, between two or more polypeptide sequences or polynucleotide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide.

A variant polypeptide may have substantially the same functional activity as a reference polypeptide. For example, a variant polypeptide may exhibit one or more biological activities associated with binding a ligand and/or binding DNA at a specific binding site.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, J. et al. (1989) Molecular Cloning: A Laboratory Manual, 2nd ed., vol. 13, Cold Spring Harbor Press, Plainview N.Y. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The term “cDNA” as used herein refers to all polynucleotides that share the arrangement of sequence elements found in native mature mRNA species, where sequence elements are exons and 5′ and 3′ non-coding regions. Normally mRNA species have contiguous exons, with the intervening introns, when present, being removed by nuclear RNA splicing, to create a continuous open reading frame encoding the protein.

The term “homologous” as used herein in reference to polynucleotides and polynucleotide sequences is intended to mean obtainable from the same biological species, i.e. a first and second polynucleotide sequence are homologous when they are obtainable from the same biological species, and conversely, a first and second polynucleotide sequence are non-homologous when they are obtainable or obtained from two different biological species.

The term “in vitro” as used herein refers to the performance of a biochemical reaction outside a living cell, including, for example, in a microwell plate, a tube, a flask, a tank, a reactor and the like, for example a reaction to form an alkaloid compound.

The term “in vivo” as used herein refers to the performance of a biochemical reaction within a living cell, including, for example, a microbial cell, or a plant cell, for example to form an alkaloid compound.

The term “substantial sequence identity” between polynucleotide or polypeptide sequences refers to polynucleotide or polypeptide comprising a sequence that has at least 80% sequence identity, preferably at least 85%, more preferably at least 90% and most preferably at least 95%, even more preferably, at least 96%, 97%, 98% or 99% sequence identity, however in each case less than 100%, compared to a reference polynucleotide sequence using the programs.

The terms “O-methyltransferase”, or “OMT”, which may be used interchangeably herein, refer to any and all enzymes comprising a sequence of amino acid residues which is (i) substantially identical to the amino acid sequences constituting any OMT polypeptide set forth herein, including, for example, SEQ. ID NO: 1, or (ii) encoded by a nucleic acid sequence capable of hybridizing under at least moderately stringent conditions to any nucleic acid sequence encoding any OMT polypeptide set forth herein, but for the use of synonymous codons.

“Transformation” describes a process by which exogenous DNA is introduced into a recipient cell. Transformation may occur under natural or artificial conditions according to various methods well known in the art and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

“Substantially isolated or purified” nucleic acid or amino acid sequences are contemplated herein. The term “substantially isolated or purified” refers to nucleic acid or amino acid sequences that are removed from their natural environment, and are at least 60% free, preferably at least 75% free, and more preferably at least 90% free, even more preferably at least 95% free from other components with which they are naturally associated.

### Engineered Methyltransferases

Disclosed herein is a non-naturally occurring methyltransferase, wherein said methyltransferase can methylate more than two positions in a benzylisoquinoline alkaloid (BIA) molecule. BIAs and methods of producing them are known in the art. For example, Valentic et al. (ACS Catal. 2020, 10, 4497-4509) discloses a synthetic pathway for the manufacture of a BIA. Because microbial systems are more genetically tractable and easier to cultivate than the native plant producers, microbial BIA production platforms can provide new avenues for the generation of currently inaccessible BIA biosynthetic intermediates and unnatural BIA derivatives through pathway and enzyme engineering.

One key class of enzymes required in the maturation of a BIA scaffold into a final product are methyltransferases. As disclosed in Valentic et al, plant BIA O-methyltransferases (OMTs) are responsible for the regiospecific O-methylation of BIA scaffolds at distinct sites, which is often required for subsequent downstream enzymatic processing. In most cases, plant BIA OMTs function as homodimers that exhibit stringent substrate scope and methylation regional specificities and are employed only once in a given biosynthetic pathway.

Although several classes of plant BIA OMTs have been discovered and functionally characterized, many plant OMTs involved in the biosynthesis of medicinally relevant BIAs or BIA derivatives remain undiscovered or uncharacterized. Recently, several multifunctional OMT orthologues involved in BIA metabolism were discovered from Glaucium flavum (GFLOMT1-6) and Eschscholzia californica (EcG3OMT) and were shown to accept a variety of BIA substrates in vitro (Plant physiol. 2015. October; 169(2):1127-40).

These examples of substrate promiscuity in natural OMTs demonstrate the potential to use protein engineering to alter OMT substrate preference or regioselectivity toward minor or even entirely novel products.

It was therefore reasoned that one of these promiscuous methyltransferases could be evolved to methylate several BIA oxygen positions and effectively reduce the number of enzymes needed to biosynthesize more advanced BIA products. An example of such an enzyme can be found in Example 1. Therefore, disclosed herein are methyltransferases that can methylate 2, 3, 4, or more methylation sites in a composition.

The terms “alkaloid” or “alkaloid compound”, as may be used interchangeably herein, refers to naturally occurring chemical compounds containing basic nitrogen atoms, and derivatives and analogues thereof, including, but not limited to compounds belonging to the pyridine group (for example, piperine and nicotine); the pyrrolidine group (for example, hygrine, cuscohygrine, nicotine); the tropane group (for example, atropine, and cocaine); the quinoline group (for example, quinine, quinidine, dihydroquinine, dihydroquinidine, strychnine); the isoquinoline group; the phenanthrene alkaloid group; the phenethyl amine group (for example, mescaline, ephedrine, dopamine); the indole group which includes tryptaniines (for example, serotonin), ergolines (for example, ergine, ergotamine, lysergic acid, LSD), beta-carbolines (for example, harmine, harmaline, tetrahydroharmine), yohimbans (for example, reserpine, yohimbine), vinca alkaloids (for example, vinblastine, vincristine), mitragyna speciosa alkaloids (for example, mitragynine, 7-hydroxymitragynine), tabernanthe iboga alkaloids (for example, ibogaine, voacangine, coronaridine, 18-methoxycoronaridine), strychnos nuxvomica alkaloids (for example, strychnine, brucine); the purine group (for example, xanthines: caffeine, theobromine, theophylline); the terpenoid group which include aconite alkaloids (aconitine), steroid alkaloids (containing a steroid skeleton in a nitrogen containing structure, for example, solanum (for example, potato and tomato) alkaloids (solanidine, solanine, chaconine), veratrum alkaloids (veratramine, cyclopamine, cycloposine, jervine, muldamine), newt alkaloids (samandarin), and others (for example, conessine); quaternary ammonium compound group (for example, muscarine, choline, neurine); and miscellaneous alkaloids such as, for example, capsaicin, cynarin, phytolaccine, and phytolacco toxin. U.S. Pat. No. 10,487,345, herein incorporated by reference in its entirety, discloses the formulas for these alkaloids.

Specifically disclosed herein are benzylisoquinoline alkaloids (BIAs). BIAs are a structurally diverse group of plant specialized metabolites. Hagel et al. (Plant and Cell Physiology, Volume 54, Issue 5, May 2013, Pages 647-672, hereby incorporated by reference in its entirety for its disclosure concerning benzylisoquinoline alkaloids) discloses multiple BIAs. Disclosed herein are methyltransferases capable of methylating BIAs, as well as biosynthetic precursors, intermediates, and metabolites thereof. Examples of these BIAs, biosynthetic precursors, intermediates, and metabolites thereof include but not limited to papaverine and tetrahydropapaverine, protopine, magnoflorine, noscapine, reticuline, sanguinarine, dihydrosanguinarine, cularine, rhoeadine, duaricine, pavine, isopavine, protoberberine, berberine, a benzophenanthridine alkaloid, thebaine, cheilanthifoline, stylopine, cis-N-methylstylopine, salutaridine, salutaridinol, salutaridinol-7-O-acetate, (S)-canadine, oripavine, codeinone, neopine, neomorphine, morphine, promorphinin, morphinan, codeine, hydromorphone, hydrocodone, codeine, phthalideisoquinoline, 1-benzylisoquinoline, norlaudanosoline, reticuline, norreticuline, norcoclaurine, coclaurine, benzophenathridine, and bisbenzylisoquinoline. In one specific embodiment, the BIA is tetrahydropapaverine (THP) or norlaudanosoline (NOR).

The methyltransferase can be engineered from a known, or naturally occurring, methyltransferase. For example, the engineered methyltransferase can be derived from naturally occurring O-methyltransferases, such as GfOMT1 (SEQ. ID NO: 1), GfOMT2 (SEQ. ID NO: 2), GfOMT6 (SEQ. ID NO: 3) and GfOMT7 (SEQ. ID NO: 4). Importantly, the engineered methyltransferase has been modified to methylate more than one methylation site in the BIA simultaneously. By simultaneously is meant effectively at the same time, without requiring a separate reaction.

The engineered methyltransferase disclosed herein can be about 80, 81, 82, 83, 84, 85, 86, 87, 88, 89, 90, 91, 92, 93, 94, 95, 96, 97, 98, or 99% identical to SEQ ID NO: 1. Viewed another way, the engineered methyltransferase can have 1, 2, 3, 4, 5, 6, 7, 8, 9, 10, 11, 12, 13, 14, 15, 16, 17, 18, 19, or 20 or more amino acid variations when compared to SEQ ID NO 1. Such variations can be substitutions, deletions, or insertions. For example, disclosed herein is an engineered methyltransferase comprising SEQ ID NO: 6, wherein SEQ ID NO: 6 varies from SEQ ID NO: 1 in the following positions: T178A, K146R, W22L, I258V, L177M, A291V, and M121V.

Also disclosed herein is a nucleic acid encoding the methyltransferases disclosed herein, as well as host cells. The host cells may also be modified to possess one or more genetic alterations (nucleic acids) to accommodate the heterologous coding sequences. Alterations of the native host genome include, but are not limited to, modifying the genome to reduce or ablate expression of a specific enzyme that may interfere with the desired pathway. The presence of such native enzymes may rapidly convert one of the intermediates or final products of the pathway into a metabolite or other compound that is not usable in the desired pathway. Thus, if the activity of the native enzyme were reduced or altogether absent, the produced intermediates would be more readily available for incorporation into the desired product. Genetic alterations may also include modifying the promoters of endogenous genes to increase expression and/or introducing additional copies of endogenous genes. Examples of this include the construction/use of strains which overexpress the endogenous yeast NADPH-P450 reductase CPR1 to increase activity of heterologous P450 enzymes, or the overexpression of the endogenous S-adenosylmethionine synthetase for higher S-adenosylmethionine cofactor generation. In addition, endogenous enzymes such as ARO8, 9, and 10, which are directly involved in the synthesis of intermediate metabolites, may also be overexpressed.

The heterologous coding sequences of the present invention are sequences that encode enzymes, either wild-type or equivalent sequences, which are normally responsible for the production of BIAs in plants. The enzymes for which the heterologous sequences code can be any of the enzymes in the BIA pathway and can be from any known source. The choice and number of enzymes encoded by the heterologous coding sequences for the particular synthetic pathway should be chosen based upon the desired product. For example, the host cells of the present invention may comprise at least 1, 2, 3, 4, 5, 6, 7, 8, 9, 10, 11, 12, 13, 14, 15 or more heterologous coding sequences (nucleic acids). Methods of preparing BIAs using these modified cells are discussed in more detail below.

### Methods of Preparing BIAs

Disclosed herein is a method of preparing a benzylisoquinoline alkaloid (BIA) composition, wherein the benzylisoquinoline alkaloid (BIA) composition requires methylation in its final form, the method comprising: culturing a host cell under suitable conditions, wherein the host cell comprises nucleic acid encoding a non-naturally occurring methyltransferase; exposing the methyltransferase to the precursor of the BIA composition; and allowing the methyltransferase to methylate the precursor to the BIA composition, wherein the methyltransferase methylates more than two positions in the precursor of the BIA composition, thereby producing a methylated composition of interest.

As mentioned above, disclosed herein is a host cell that produces one or more BIAs of interest. Any convenient cells may be utilized in the subject host cells and methods. In some cases, the host cells are non-plant cells. In some instances, the host cells may be characterized as microbial cells. In certain cases, the host cells are insect cells, mammalian cells, bacterial cells, or yeast cells.

Host cells of interest include, but are not limited to, bacterial cells, such as Bacillus subtilis, Escherichia coli, Streptomyces and Salmonella typhimuium cells, insect cells such as Drosophila melanogaster S2 and Spodoptera frugiperda Sf9 cells, and yeast cells such as Saccharomyces cerevisiae, Schizosaccharomyces pombe, and Pichia pastoris cells. In some embodiments, the host cells are yeast cells or E. coli cells. In some cases, the host cell is a yeast cell. In some instances, the host cell is from a strain of yeast engineered to produce a BIA of interest. In certain embodiments, the yeast cells may be of the species Saccharomyces cerevisiae (S. cerevisiae). In certain embodiments, the yeast cells may be of the species Schizosaccharomyces pombe. In certain embodiments, the yeast cells may be of the species Pichia pastoris. Yeast is of interest as a host cell because cytochrome P450 proteins, which are involved in some biosynthetic pathways of interest, are able to fold properly into the endoplasmic reticulum membrane so that their activity is maintained.

Yeast strains of interest that find use in the invention include, but are not limited to, CEN.PK (Genotype: MATa/α ura3-52/ura3-52 trp1-289/trp1-289 leu2-3_112/leu2-3_112 his3 Δ1/his3 Δ1 MAL2-8C/MAL2-8C SUC2/SUC2), S288C, W303, D273-10B, X2180, A364A, Σ1278B, AB972, SK1, and FL100. In certain cases, the yeast strain is any of S288C (MATα; SUC2 mal mel gal2 CUP1 flo1 flo8-1 hap1), BY4741 (MATα; his3Δ1; leu2Δ0; met15Δ0; ura3Δ0), BY4742 (MATα; his3Δ1; leu2Δ0; lys2Δ0; ura3Δ0), BY4743 (MATa/MATα; his3Δ1/his3Δ1; leu2Δ0/leu2Δ0; met15Δ0/MET15; LYS2/lys2Δ0; ura3Δ0/ura3Δ0), and WAT11 or W(R), derivatives of the W303-B strain (MATa; ade2-1; his3-11, -15; leu2-3, -112; ura3-1; canR; cyr+) which express the Arabidopsis thaliana NADPH-P450 reductase ATR1 and the yeast NADPH-P450 reductase CPR1, respectively. In another embodiment, the yeast cell is W303alpha (MATa; his3-11, 15 trp1-1 leu2-3 ura3-1 ade2-1). The identity and genotype of additional yeast strains of interest may be found at EUROSCARF (web.uni-frankfurt.de/fb15/mikro/euroscarf/col_index.html).

The host cells may be engineered to include one or more modifications (such as two or more, three or more, four or more, five or more, or even more modifications) that provide for the production of BIAs of interest. In some cases, by modification is meant a genetic modification, such as a mutation, addition, or deletion of a gene or fragment thereof, or transcription regulation of a gene or fragment thereof. In some cases, the one or more (such as two or more, three or more, or four or more) modifications is selected from: a feedback inhibition alleviating mutation in a biosynthetic enzyme gene native to the cell; a transcriptional modulation modification of a biosynthetic enzyme gene native to the cell; an inactivating mutation in an enzyme native to the cell; and a heterologous coding sequence that encodes an enzyme. A cell that includes one or more modifications may be referred to as a modified cell.

A modified cell may overproduce one or more precursor BIA, BIA, or modified BIA molecules. By overproduce is meant that the cell has an improved or increased production of a BIA molecule of interest relative to a control cell (e.g., an unmodified cell). By improved or increased production is meant both the production of some amount of the BIA of interest where the control has no BIA precursor production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some BIA of interest production.

In some cases, the host cell is capable of producing an increased amount of tetrahydropapaverine relative to a control host cell that lacks the modified methyltransferase described herein In certain instances, the increased amount of tetrahydropapaverine is about 10% or more relative to the control host cell, such as about 20% or more, about 30% or more, about 40% or more, about 50% or more, about 60% or more, about 80% or more, about 100% or more, 2-fold or more, 5-fold or more, or even 10-fold or more relative to the control host cell.

In some embodiments of the host cell, when the cell includes one or more heterologous coding sequences that encode one or more enzymes, it includes at least one additional modification selected from the group consisting of: a feedback inhibition alleviating mutations in a biosynthetic enzyme gene native to the cell; a transcriptional modulation modification of a biosynthetic enzyme gene native to the cell; and an inactivating mutation in an enzyme native to the cell. In certain embodiments of the host cell, when the cell includes one or more feedback inhibition alleviating mutations in one or more biosynthetic enzyme genes native to the cell, it includes a least one additional modification selected from the group consisting of: a transcriptional modulation modification of a biosynthetic enzyme gene native to the cell; an inactivating mutation in an enzyme native to the cell; and a heterologous coding sequence that encode an enzyme. In some embodiments of the host cell, when the cell includes one or more transcriptional modulation modifications of one or more biosynthetic enzyme genes native to the cell, it includes at least one additional modification selected from the group consisting of: a feedback inhibition alleviating mutation in a biosynthetic enzyme gene native to the cell; an inactivating mutation in an enzyme native to the cell; and a heterologous coding sequence that encodes an enzyme. In certain instances of the host cell, when the cell includes one or more inactivating mutations in one or more enzymes native to the cell, it includes at least one additional modification selected from the group consisting of: a feedback inhibition alleviating mutation in a biosynthetic enzyme gene native to the cell; a transcriptional modulation modification of a biosynthetic enzyme gene native to the cell; and a heterologous coding sequence that encodes an enzyme.

Also disclosed herein is a kit comprising: a non-naturally occurring methyltransferase, wherein said methyltransferase can methylate more than two positions in a benzylisoquinoline alkaloid (BIA) molecule. The kit can include one or more additional components as outlined above.

### EXAMPLES

### Example 1: Tetrahydropapaverine Biosynthesis Enabled by a Novel Alkaloid Sensor

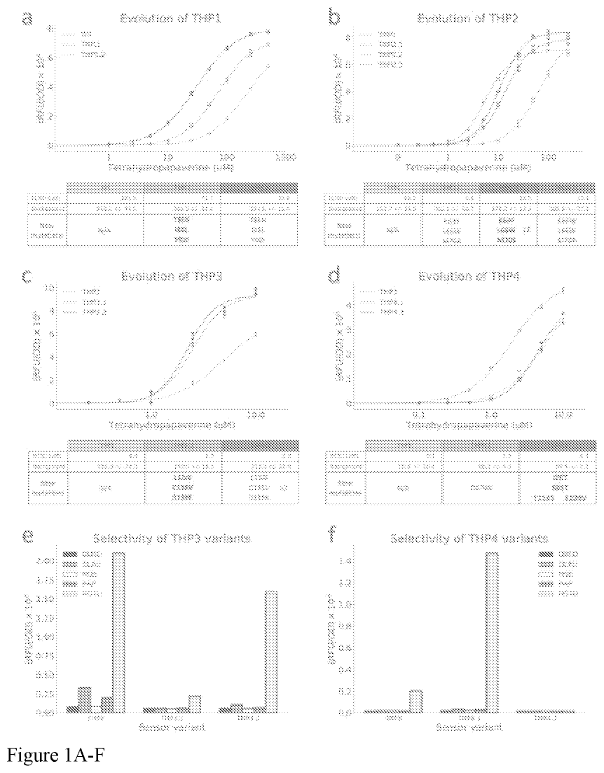

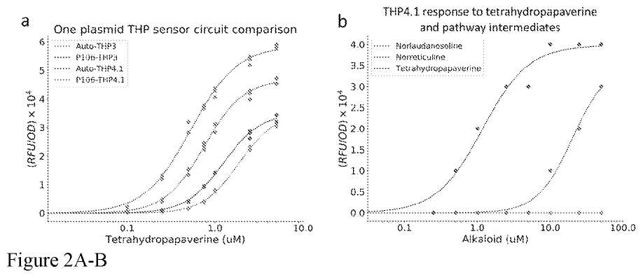

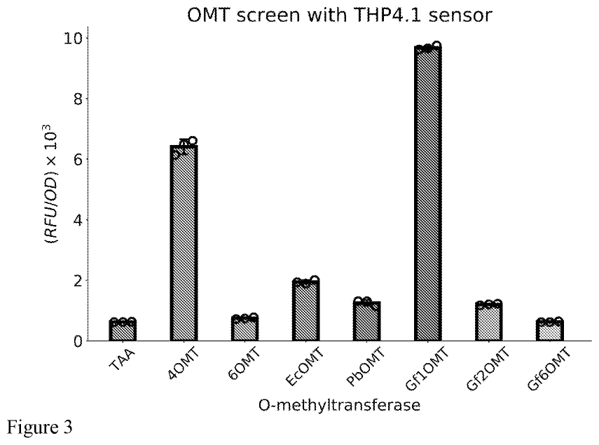

Leveraging highly engineered alkaloid biosensors, a THP reporter plasmid (pThpR) (FIG. 1d) was used to screen a panel of methyltransferases. The THP sensor variant and expression were modified to maximize the sensitivity of the reporter plasmid (FIG. 2a). Since the THP sensor employed (THP4.2) was somewhat responsive to a semi-methylated intermediate (norreticuline) but not the unmethylated substrate, this circuit reported on general methylation activity, likely favoring more completely methylated derivatives (FIG. 2b). One methyltransferase, GfOMT1 from Glaucium flavum, produced the highest fluorescent signal in E. coli bearing pThpR when cultured with norlaudanosoline (NOR) and was used as the starting point for evolution (FIG. 3).

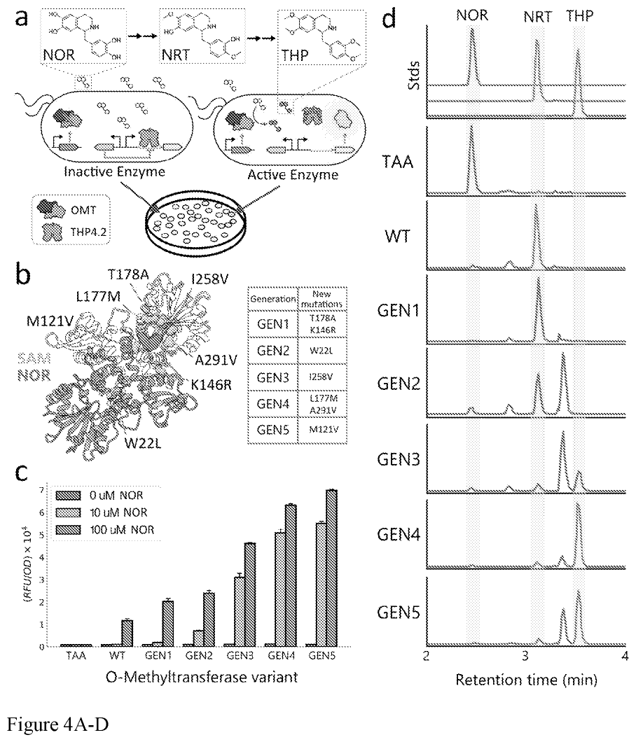

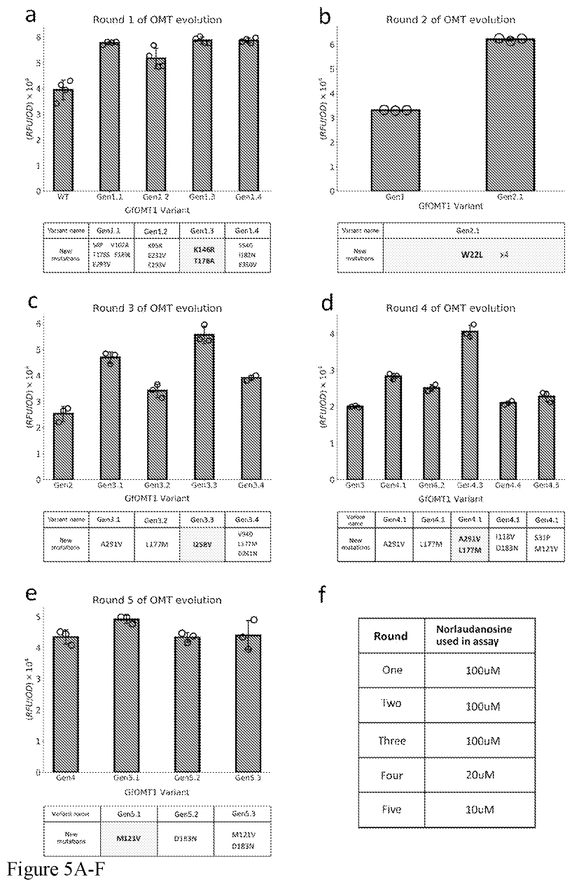

Error-prone libraries of GfOMT1 were generated with an average of two mutations relative to the template and co-transformed with pThpR. Highly fluorescent colonies were screened on solid media supplemented with NOR (FIG. 4a). The resulting enzyme variants were subcloned and re-phenotyped (FIG. 5), and the best performing variant was then used as the template for the next round of evolution.

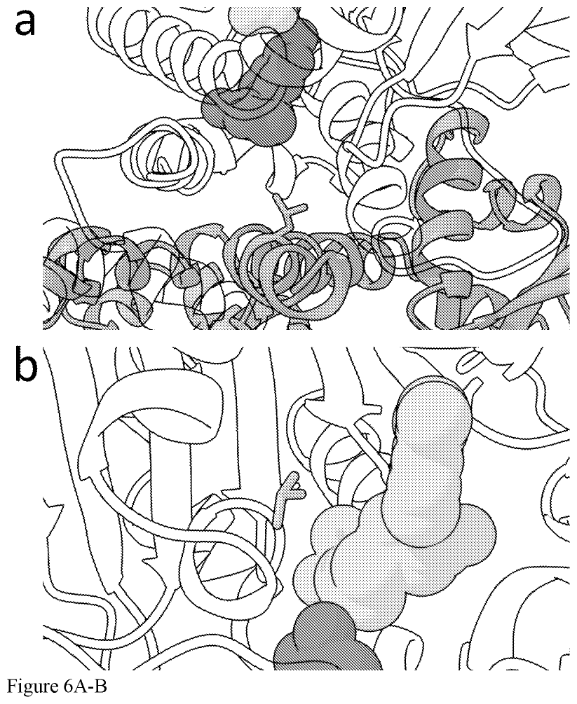

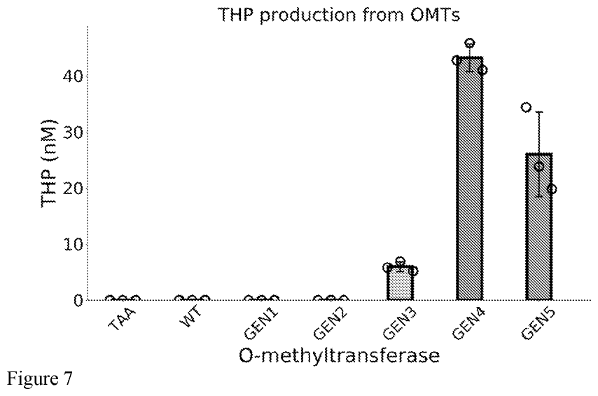

Over five rounds of evolution a GfOMT1 variant with seven substitutions (GEN5) produced a 6- and 47-fold increase in fluorescent signal using pThpR compared to wild-type GfOMT1 when cultured with 100 μM or 10 μM of NOR, respectively (FIG. 4b-c). LC/MS analysis of enzymatic reactions showed that later OMT generations successively methylate more positions on NOR and yield increasing amounts of THP. Notably, the W22L mutation that occurred in GEN2 is thought to expand the substrate binding pocket and enable production of the trimethylated intermediate (FIG. 6a) whereas the I258V mutation from GEN3 is adjacent to the expected S-adenosyl-methionine binding site and enables complete methylation to THP (FIG. 6b). Interestingly, the GEN4 variant produced THP more selectively and efficiently than GEN5, despite both producing a similar fluorescent response with the pThpR reporter (FIG. 4c-d) (FIG. 7). This may be due to a higher accumulation of a semi-methylated intermediate produced by GEN5 that the THP sensor also responds to. Paired with the highly sensitive pThpR reporter, the GEN4 methyltransferase can accelerate further optimization of upstream pathway genes and enable the complete biosynthesis of THP.

| | Observed EIC Areas | | | | |
|---|---|---|---|---|---|
| | Sample | | | | |
| | NOR-4OH | 3OH | NRT-2OH | 1OH | THP-0OH |
| | (288.1230 | (302.1387 | (316.1543 | (330.1700 | (344.1856 |
| | m/z) | m/z) | m/z) | m/z) | m/z) |
| | Retention Time (min.) | | | | |
| | 2.46 | 2.85 | 3.14 | 3.41 | 3.58 |
| 1(TAA_1) | 1,900,714.32 | 0.00 | 0.00 | 0.00 | 732,062.09 |
| 2(TAA_2) | 1,915,746.57 | 0.00 | 0.00 | 0.00 | 739,719.41 |
| 3(TAA_3) | 2,429,528.49 | 0.00 | 0.00 | 0.00 | 683,951.95 |
| 4(WT_1) | 292,886.91 | 838,007.19 | 5,016,094.31 | 0.00 | 715,848.03 |
| 5(WT_2) | 183,019.07 | 678,233.01 | 4,455,448.19 | 0.00 | 726,021.76 |
| 6(WT_3) | 553,463.54 | 956,624.78 | 5,512,157.20 | 0.00 | 724,935.11 |
| 7(Gen1_1) | 127,968.53 | 262,284.74 | 5,028,466.49 | 0.00 | 662,206.48 |
| 8(Gen1_2) | 189,597.68 | 357,195.05 | 5,328,381.15 | 0.00 | 685,462.09 |
| 9(Gen1_3) | 69,624.59 | 194,528.60 | 4,578,048.19 | 0.00 | 627,833.84 |
| 10(Gen2_1) | 641,245.71 | 730,076.27 | 2,721,068.90 | 4,013,082.04 | 768,144.56 |
| 11(Gen2_2) | 251,793.95 | 485,465.06 | 1,499,029.94 | 4,748,195.68 | 825,357.07 |
| 12(Gen2_3) | 457,481.05 | 582,826.88 | 2,227,163.03 | 4,367,581.54 | 796,201.94 |
| 13(Gen3_1) | 304,453.82 | 474,632.34 | 603,324.03 | 6,336,545.51 | 2,220,655.54 |
| 14(Gen3_2) | 259,191.32 | 327,542.61 | 565,407.61 | 6,114,959.98 | 2,469,776.82 |
| 15(Gen3_3) | 388,680.89 | 444,919.64 | 622,087.57 | 6,641,956.94 | 2,075,163.88 |
| 16(Gen4_1) | 242,876.32 | 0.00 | 639,571.41 | 1,994,412.63 | 10,694,641.10 |
| 17(Gen4_2) | 139,427.97 | 0.00 | 488,824.56 | 1,353,523.79 | 11,406,507.42 |
| 18(Gen4_3) | 249,690.78 | 0.00 | 684,700.15 | 2,054,059.80 | 10,304,271.54 |
| 19(Gen5_1) | 33,523.35 | 55,140.14 | 377,855.13 | 3,057,004.77 | 8,779,382.47 |
| 20(Gen5_2) | 159,548.08 | 235,055.43 | 565,294.52 | 4,882,357.32 | 6,352,723.48 |
| 21(Gen5_3) | 385,426.52 | 238,451.26 | 608,492.27 | 5,854,903.52 | 5,431,738.76 |
| 22(NOR_50 nM_STD) | 1,674,119.68 | 0.00 | 0.00 | 0.00 | 650,085.33 |
| 23(NRT_50 nM_STD) | 0.00 | 0.00 | 6,704,519.34 | 0.00 | 659,703.80 |
| 24(THP_10 nM_STD) | 0.00 | 0.00 | 0.00 | 0.00 | 3,178,252.46 |
| 25(THP_50 nM_STD) | 0.00 | 0.00 | 0.00 | 0.00 | 11,969,036.43 |

Table 1 shows observed extracted ion chromatogram areas for all controls, standards, and enzymatic reactions. The top two rows indicate the ml/z ratio and retention time for compounds of interest (norlaudanosoline (NOR-4OH), 6-O-Methylnorlaudanosoline (3OH), norreticuline (NRT-2OH), norlaudanine (1OH), and tetrahydropapaverine (THP-0OH). All sample measurements were performed in biological triplicate.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the claims.

SEQUENCES
>GfOMT1
SEQ. ID. NO: 1
MGVSDNKPESQEVDIKAQAHLWNIIYGFADSLVLRCAVEIGIADIIKSNN
GSISVTELASKLPITNVNSDNLYRVLRYLVHMGILKEVSDSNEVKLYSLQ
PVATLLLRDAERSMVPIILGMTQKDFMIPWHFMKEGLGNDTTAFEKGMGM
TIWQYLEGHPEQSNLFNEGMAGETRLLTKSLIDGCRDTFEGLTSLCDVGG
GNGTTIKGIYDAFPQIKCSVYDLPHVIASSPEHPNIERIPGDMFKSVPSA
QAILLKLILHDWTDEECVNILIKCREAVPKDTGKVIIVDVALEEESQHEL
TKTRLILDIDMLVNTGGRERSEDDWEKLLKRAGFRGHKIRHIAAIQSVIE
AFP
>GfOMT_MUT1
SEQ. ID. NO: 2
MGVSDNKPESQEVDIKAQAHLWNIIYGFADSLVLRCAVEIGIADIIKSNN
GSISVTELASKLPITNVNSDNLYRVLRYLVHMGILKEVSDSNEVKLYSLQ
PVATLLLRDAERSMVPIILGMTQKDFMIPWHFMKEGLGNDTTAFERGMGM
TIWQYLEGHPEQSNLFNEGMAGETRLLAKSLIDGCRDTFEGLTSLCDVGG
GNGTTIKGIYDAFPQIKCSVYDLPHVIASSPEHPNIERIPGDMFKSVPSA
QAILLKLILHDWTDEECVNILIKCREAVPKDTGKVIIVDVALEEESQHEL
TKTRLILDIDMLVNTGGRERSEDDWEKLLKRAGFRGHKIRHIAAIQSVIE
AFP
>GfOMT_MUT2
SEQ. ID. NO: 3
MGVSDNKPESQEVDIKAQAHLLNIIYGFADSLVLRCAVEIGIADIIKSNN
GSISVTELASKLPITNVNSDNLYRVLRYLVHMGILKEVSDSNEVKLYSLQ
PVATLLLRDAERSMVPIILGMTQKDFMIPWHFMKEGLGNDTTAFERGMGM
TIWQYLEGHPEQSNLFNEGMAGETRLLAKSLIDGCRDTFEGLTSLCDVGG
GNGTTIKGIYDAFPQIKCSVYDLPHVIASSPEHPNIERIPGDMFKSVPSA
QAILLKLILHDWTDEECVNILIKCREAVPKDTGKVIIVDVALEEESQHEL
TKTRLILDIDMLVNTGGRERSEDDWEKLLKRAGFRGHKIRHIAAIQSVIE
AFP
>GfOMT_MUT3
SEQ. ID. NO: 4
MGVSDNKPESQEVDIKAQAHLLNIIYGFADSLVLRCAVEIGIADIIKSNN
GSISVTELASKLPITNVNSDNLYRVLRYLVHMGILKEVSDSNEVKLYSLQ
PVATLLLRDAERSMVPIILGMTQKDFMIPWHFMKEGLGNDTTAFERGMGM
TIWQYLEGHPEQSNLFNEGMAGETRLLAKSLIDGCRDTFEGLTSLCDVGG
GNGTTIKGIYDAFPQIKCSVYDLPHVIASSPEHPNIERIPGDMFKSVPSA
QAILLKLVLHDWTDEECVNILIKCREAVPKDTGKVIIVDVALEEESQHEL
TKTRLILDIDMLVNTGGRERSEDDWEKLLKRAGFRGHKIRHIAAIQSVIE
AFP
>GfOMT_MUT4
SEQ. ID. NO: 5
MGVSDNKPESQEVDIKAQAHLLNIIYGFADSLVLRCAVEIGIADIIKSNN
GSISVTELASKLPITNVNSDNLYRVLRYLVHMGILKEVSDSNEVKLYSLQ
PVATLLLRDAERSMVPIILGMTQKDFMIPWHFMKEGLGNDTTAFERGMGM
TIWQYLEGHPEQSNLFNEGMAGETRLMAKSLIDGCRDTFEGLTSLCDVGG
GNGTTIKGIYDAFPQIKCSVYDLPHVIASSPEHPNIERIPGDMFKSVPSA
QAILLKLVLHDWTDEECVNILIKCREAVPKDTGKVIIVDVALEEESQHEL
TKTRLILDIDMLVNTGGRERSEDDWEKLLKRAGFRGHKIRHIAAIQSVIE
AFP
>GfOMT_MUT5
SEQ. ID. NO: 6
MGVSDNKPESQEVDIKAQAHLLNIIYGFADSLVLRCAVEIGIADIIKSNN
GSISVTELASKLPITNVNSDNLYRVLRYLVHMGILKEVSDSNEVKLYSLQ
PVATLLLRDAERSMVPIILGVTQKDFMIPWHFMKEGLGNDTTAFERGMGM
TIWQYLEGHPEQSNLFNEGMAGETRLMAKSLIDGCRDTFEGLTSLCDVGG
GNGTTIKGIYDAFPQIKCSVYDLPHVIASSPEHPNIERIPGDMFKSVPSA
QAILLKLVLHDWTDEECVNILIKCREAVPKDTGKVIIVDVVLEEESQHEL
TKTRLILDIDMLVNTGGRERSEDDWEKLLKRAGFRGHKIRHIAAIQSVIE
AFP

### REFERENCES

1. (Ro, DK., Paradise, E., Ouellet, M. et al. Production of the antimalarial drug precursor artemisinic acid in engineered yeast. Nature 440, 940-943 (2006).
2. Luo, X., Reiter, M. A., d'Espaux, L. et al. Complete biosynthesis of cannabinoids and their unnatural analogues in yeast. Nature 567, 123-126 (2019).
3. Science. 2015 Sep. 4; 349(6252): 1095-1100.
4. Nakagawa, A. et al. Total biosynthesis of opiates by stepwise fermentation using engineered Escherichia coli. Nat. Commun. 7:10390 doi: 10.1038/ncomms10390 (2016).
5. Srinivasan, P., Smolke, C. D. Biosynthesis of medicinal tropane alkaloids in yeast. Nature 585, 614-619 (2020).
6. https://dio.org/10.1016/j_ymben.2011.09.002
7. Metab Eng. 2021 January; 63:102-125. doi: 10.1016/j.ymben.2020.09.004. Epub 2020 Oct. 2.
8. Transcription factor-based biosensors: a molecular-guided approach for natural product engineering. Curr Opin Biotechnol. 2021. doi: 10.1016/j.copbio.2021.01.008
8*. Genetic Biosensor Design for Natural Product Biosynthesis in Microorganisms. 2020. Trends in Biotechnology.
8**. Hanko, E. K. R., Paiva, A. C., Jonczyk, M. et al. A genome-wide approach for identification and characterisation of metabolite-inducible systems. Nat Commun 11, 1213 (2020).
9. Della Corte, D., van Beek, H. L., Syberg, F. et al. Engineering and application of a biosensor with focused ligand specificity. Nat Commun 11, 4851 (2020)
9* Developing a highly efficient hydroxytyrosol whole-cell catalyst by de-bottlenecking rate-limiting steps. Nature Communications.
9** Evolution-guided engineering of small-molecule biosensors. Nucleic Acids Research.
9 *** Switching the Ligand Specificity of the Biosensor XylS from meta to para-Toluic Acid through Directed Evolution Exploiting a Dual Selection System. ACS Synthetic Biology.
10. Protein engineers turned evolutionists—the quest for the optimal starting point. Current Opinion in Biotechnology. 2019. December; 60(12):46-52
11. 2015. Expanding the Enzyme Universe: Accessing Non-Natural Reactions by Mechanism-Guided Directed Evolution.
12. 2012. Directed enzyme evolution: beyond the low-hanging fruit
13. 2010. MD recognition by MDR gene regulators. Herschel Wade. Current Opinion Structural Biology. Volume 20, Issue 4, August 2010, Pages 489-496
14. Improving key enzyme activity in phenylpropanoid pathway with a designed biosensor. Metabolic Engineering. Volume 40, March 2017, Pages 115-123
15. Regulatory control circuits for stabilizing long-term anabolic product formation in yeast. Metab Eng. 2020 September; 61:369-380. doi: 10.1016/j.ymben.2020.07.006. Epub 2020 Jul. 24.
16. Complete biosynthesis of noscapine and halogenated alkaloids in yeast. PNAS. 2018 Apr. 24; 115(17).
17. Structure-Guided Engineering of a Scoulerine 9-O-Methyltransferase Enables the Biosynthesis of Tetrahydropalmatrubine and Tetrahydropalmatine in Yeast. Smolke. ACS Catalysis.
18. Genomic mining of prokaryotic repressors for orthogonal logic gates. Voigt. 2014. Nature Chemical Biology.
18*. nalD Encodes a Second Repressor of the mexAB-oprM Multidrug Efflux Operon of Pseudomonas aeruginosa. 2006. J Bacteriology.
18**. Cloning and characterization of SmeT, a repressor of the Stenotrophomonas maltophilia multidrug efflux pump SmeDEF. 2002. Antimicrob Agents Chemother.
19. The crystal structure of multidrug-resistance regulator RamR with multiple drugs. Nature Communications. 2013.
20. Bleomycin resistance conferred by a drug-binding protein. FEBS Letter. 1988.
21. Accelerating the semisynthesis of alkaloid-based drugs through metabolic engineering. 2017. Nature Chemical Biology.
22. 3′O-Methyltransferase, Ps3′OMT, from opium poppy: involvement in papaverine biosynthesis. 2019. Plant Cell Reports.
23. Fermentative production of tetrahydropapaverine and its derivatives using Escherichia coli. Akira NAKAGAWA.
24. Isolation and Characterization of O-methyltransferases Involved in the Biosynthesis of Glaucine in Glaucium flavum. 2015 Facchini. Plant Physiology.
25. Synthetic biology strategies for microbial biosynthesis of plant natural products. 2019. Smolke. Nature Communications.
26. Design of an in vitro biocatalytic cascade for the manufacture of islatravir. 2019. Science.
27. The nature of chemical innovation: new enzymes by evolution. 2015. Q Rev Biophys.
28. Crystal structure of the multidrug resistance regulator RamR complexed with bile acids. 2019. Sci Rep
29. Isoquinolines: Important Cores in Many Marketed and Clinical Drugs. 2021. Anticancer Agents Med Chem.
30. Privileged Scaffolds for Library Design and Drug Discovery. 2015. Curr Opin Chem Biol.
31. Dynamic control of toxic natural product biosynthesis by an artificial regulatory circuit. 2020. Metabolic Engineering
32. Synthetic addiction extends the productive life time of engineered Escherichia coli populations. PNAS. 2018.
33. An ingestible bacterial-electronic system to monitor gastrointestinal health. 2018. Science.
34. Cell-free biosensors for rapid detection of water contaminants. 2021. Nat Biotechnol.
35. Cascaded amplifying circuits enable ultrasensitive cellular sensors for toxic metals. 2019. Nat Chem Biol.
36. Harnessing the central dogma for stringent multi-level control of gene expression. 2021. Nat Comm.
37. A suppressor tRNA-mediated feedforward loop eliminates leaky gene expression in bacteria. 2021. NAR.
38. Regulation by tetracycline of gene expression in Saccharomyces cerevisiae. 1997. Molecular and General Genetics MGG.
39. Tight control of gene expression in mammalian cells by tetracycline-responsive promoters. 1992.
40. Stringent repression and homogeneous de-repression by tetracycline of a modified CaMV 35S promoter in intact transgenic tobacco plants. 1992. Plant J.

1. A biosensor comprising an engineered substrate-promiscuous regulator, wherein said substrate-promiscuous regulator has been engineered to interact more efficiently with an input signal than does the naturally occurring substrate promiscuous regulator; and further wherein the biosensor is engineered to provide an output signal, wherein said output signal is generated in response to interaction with the input signal.

2. The biosensor of claim 1, wherein the naturally occurring regulator from which the engineered biosensor is derived is RamR of Salmonella typhimurium.

3. The biosensor of claim 1, wherein the engineered biosensor has about 97% to 99% identity to QacR (WP_001807342.1), TtgR (WP_010952495.1), SmeT (WP_005414519.1), NalD (WP_003092152.1), LmrR (WP_011834386.1), EbrR (WP_003976902), MexR (WP_003114897.1), LadR (WP_003721913.1), VceR (WP_001264144.1), MttR (WP_003693763.1), AcrR (WP_000101737), MepR (WP_000397416.1), SCO4008 (WP_011029378.1), Rv3066 (WP_003416005.1), CgmR (WP_011015249.1), CmeR (WP_002857627.1), Rv0302 (WP_003401571.1), BepR (WP_004687968.1), MexL (WP_003092468.1), TtgT (WP_012052586.1), TtgV (WP_014003968.1), LmrA (WP_003246449.1), TM_1030 (WP_010865247.1) or Bm3R1 (WP_013083972.1), or RamR (WP_000113609.1).

4. The biosensor of claim 1, wherein said input signal is a naturally occurring composition.

5. The biosensor of claim 1, wherein said input signal is a synthetic composition and is not naturally occurring.

6. The biosensor of claim 4, wherein the naturally occurring composition is a plant alkaloid.

7. The biosensor of claim 6, wherein said plant alkaloid is tetrahydropapaverine, papaverine, rotundine, glaucine, noscapine, norbelladine, or 4-o-methylnorbelladine.

8. The biosensor of claim 1, wherein the output signal is expression of a gene.

9. The biosensor of claim 1, wherein the output signal is fluorescence, luminescence, or a colorimetric signal.

10. The biosensor of claim 1, wherein the input signal is converted to the output signal by a transduction system.

11. The biosensor of claim 10, wherein the transduction system comprises a transcriptional activator or transcriptional repressor of the output signal.

12. The biosensor of claim 11, wherein the transcriptional activator or transcriptional repressor is encoded with the engineered substrate promiscuous regulator.

13. The biosensor of claim 11, wherein the transduction system comprises a promoter or operator and a regulator.

14. The biosensor of claim 1, wherein the biosensor is 90% or more identical to the naturally occurring form of the substrate promiscuous regulator.

15. The biosensor of claim 1, wherein said interaction with the input signal occurs via a covalent or a non-covalent bond.

16. The biosensor of claim 1, wherein the substrate promiscuous regulator comprises a large hydrophobic binding pocket.

17. The biosensor of claim 1, wherein the substrate promiscuous regulator is a multidrug resistance regulator.

18. A plasmid comprising a nucleic acid encoding the biosensor of claim 1.

19. The plasmid of claim 19, wherein said plasmid further comprises a nucleic acid encoding the output signal.

20. A cell comprising the plasmid of claim 18.

21. The biosensor of claim 1, wherein the biosensor is integrated into a host genome of a cell.

22. The cell of claim 20, wherein the cell is further engineered to produce a product of interest.

23. (canceled)

24. A method of making a product of interest, the method comprising
a. providing the recombinant host cell of claim 22; and
b. contacting the recombinant host cell with reagents needed to produce the product under conditions whereby a product is produced.

25. A method of engineering a substrate-promiscuous regulator to function as a biosensor, the method comprising:
a. identifying a naturally occurring substrate-promiscuous regulator;
b. engineering the naturally occurring substrate-promiscuous regulator for increased sensitivity to an input signal when compared to the naturally occurring substrate promiscuous regulator;
c. introducing into a cell:
i. nucleic acid encoding the engineered substrate-promiscuous regulator of step b), and
ii. a transduction system for providing an output signal, wherein said output signal is generated in response to interaction with the input signal;
d. exposing the cell of step c) to the input signal; and
e. detecting an output signal; wherein detection of said output signal indicates a functional biosensor.

26-36. (canceled)

37. A kit comprising a biosensor comprising an engineered substrate-promiscuous regulator, wherein said substrate-promiscuous regulator has been engineered to interact more efficiently with an input signal than does the naturally occurring substrate promiscuous regulator.

38-49. (canceled)

50. A nucleic acid comprising 97% or more identity to any one of SEQ ID NOS: 1-6.


SEQUENCE LISTING (6 sequences)

SEQ ID NO: 1 - protein, 353 aa, Artificial Sequence
  Synthetic Construct
MGVSDNKPESQEVDIKAQAHLWNIIYGFADSLVLRCAVEIGIADIIKSNNGSISVTELASKLPITNVNSDNLYRVLRYLVHMGILKEVSDSNEVKLYSLQPVATLLLRDAERSMVPIILGMTQKDFMIPWHFMKEGLGNDTTAFEKGMGMTIWQYLEGHPEQSNLFNEGMAGETRLLTKSLIDGCRDTFEGLTSLCDVGGGNGTTIKGIYDAFPQIKCSVYDLPHVIASSPEHPNIERIPGDMFKSVPSAQAILLKLILHDWTDEECVNILIKCREAVPKDTGKVIIVDVALEEESQHELTKTRLILDIDMLVNTGGRERSEDDWEKLLKRAGFRGHKIRHIAAIQSVIEAFP

SEQ ID NO: 2 - protein, 353 aa, Artificial Sequence
  Synthetic Construct
MGVSDNKPESQEVDIKAQAHLWNIIYGFADSLVLRCAVEIGIADIIKSNNGSISVTELASKLPITNVNSDNLYRVLRYLVHMGILKEVSDSNEVKLYSLQPVATLLLRDAERSMVPIILGMTQKDFMIPWHFMKEGLGNDTTAFERGMGMTIWQYLEGHPEQSNLFNEGMAGETRLLAKSLIDGCRDTFEGLTSLCDVGGGNGTTIKGIYDAFPQIKCSVYDLPHVIASSPEHPNIERIPGDMFKSVPSAQAILLKLILHDWTDEECVNILIKCREAVPKDTGKVIIVDVALEEESQHELTKTRLILDIDMLVNTGGRERSEDDWEKLLKRAGFRGHKIRHIAAIQSVIEAFP

SEQ ID NO: 3 - protein, 353 aa, Artificial Sequence
  Synthetic Construct
MGVSDNKPESQEVDIKAQAHLLNIIYGFADSLVLRCAVEIGIADIIKSNNGSISVTELASKLPITNVNSDNLYRVLRYLVHMGILKEVSDSNEVKLYSLQPVATLLLRDAERSMVPIILGMTQKDFMIPWHFMKEGLGNDTTAFERGMGMTIWQYLEGHPEQSNLFNEGMAGETRLLAKSLIDGCRDTFEGLTSLCDVGGGNGTTIKGIYDAFPQIKCSVYDLPHVIASSPEHPNIERIPGDMFKSVPSAQAILLKLILHDWTDEECVNILIKCREAVPKDTGKVIIVDVALEEESQHELTKTRLILDIDMLVNTGGRERSEDDWEKLLKRAGFRGHKIRHIAAIQSVIEAFP

SEQ ID NO: 4 - protein, 353 aa, Artificial Sequence
  Synthetic Construct
MGVSDNKPESQEVDIKAQAHLLNIIYGFADSLVLRCAVEIGIADIIKSNNGSISVTELASKLPITNVNSDNLYRVLRYLVHMGILKEVSDSNEVKLYSLQPVATLLLRDAERSMVPIILGMTQKDFMIPWHFMKEGLGNDTTAFERGMGMTIWQYLEGHPEQSNLFNEGMAGETRLLAKSLIDGCRDTFEGLTSLCDVGGGNGTTIKGIYDAFPQIKCSVYDLPHVIASSPEHPNIERIPGDMFKSVPSAQAILLKLVLHDWTDEECVNILIKCREAVPKDTGKVIIVDVALEEESQHELTKTRLILDIDMLVNTGGRERSEDDWEKLLKRAGFRGHKIRHIAAIQSVIEAFP

SEQ ID NO: 5 - protein, 353 aa, Artificial Sequence
  Synthetic Construct
MGVSDNKPESQEVDIKAQAHLLNIIYGFADSLVLRCAVEIGIADIIKSNNGSISVTELASKLPITNVNSDNLYRVLRYLVHMGILKEVSDSNEVKLYSLQPVATLLLRDAERSMVPIILGMTQKDFMIPWHFMKEGLGNDTTAFERGMGMTIWQYLEGHPEQSNLFNEGMAGETRLMAKSLIDGCRDTFEGLTSLCDVGGGNGTTIKGIYDAFPQIKCSVYDLPHVIASSPEHPNIERIPGDMFKSVPSAQAILLKLVLHDWTDEECVNILIKCREAVPKDTGKVIIVDVALEEESQHELTKTRLILDIDMLVNTGGRERSEDDWEKLLKRAGFRGHKIRHIAAIQSVIEAFP

SEQ ID NO: 6 - protein, 353 aa, Artificial Sequence
  Synthetic Construct
MGVSDNKPESQEVDIKAQAHLLNIIYGFADSLVLRCAVEIGIADIIKSNNGSISVTELASKLPITNVNSDNLYRVLRYLVHMGILKEVSDSNEVKLYSLQPVATLLLRDAERSMVPIILGVTQKDFMIPWHFMKEGLGNDTTAFERGMGMTIWQYLEGHPEQSNLFNEGMAGETRLMAKSLIDGCRDTFEGLTSLCDVGGGNGTTIKGIYDAFPQIKCSVYDLPHVIASSPEHPNIERIPGDMFKSVPSAQAILLKLVLHDWTDEECVNILIKCREAVPKDTGKVIIVDVVLEEESQHELTKTRLILDIDMLVNTGGRERSEDDWEKLLKRAGFRGHKIRHIAAIQSVIEAFP